(19) United States
(12) Patent Application Publication
(10) Pub. No.: US 2017/0265807 A1
(43) Pub. Date: Sep. 21, 2017

(54) SYSTEMS AND METHODS FOR FETAL MONITORING

(71) Applicant: Megan Stopek, Minneapolis, MN (US)

(72) Inventor: Megan Stopek, Minneapolis, MN (US)

(21) Appl. No.: 15/422,369

(22) Filed: Feb. 1, 2017

Related U.S. Application Data
(60) Provisional application No. 62/289,811, filed on Feb. 1, 2016.

(51) Int. Cl. A61B 5/00; A61B 10/00; A61B 7/00; A61B 8/08; A61B 5/01; A61B 5/15; A61B 8/12

(52) U.S. Cl. CPC A61B 5/686; A61B 5/150015; A61B 5/0084; A61B 10/0048; A61B 5/0031; A61B 8/12; A61B 8/0866; A61B 5/01; A61B 7/00; A61B 5/742

(57) ABSTRACT

In one embodiment of the disclosure, a method of monitoring a fetus in utero is disclosed that includes implanting a medical device into a patient's uterus, collecting fetal data using the medical device, and transmitting, e.g., wirelessly, the fetal data from the medical device to a receiver. In another embodiment, a medical monitoring system is disclosed that includes a first device that is implantable into a patient's uterus for collecting fetal data, and a second device that is configured and dimensioned for connection with the first device such that the fetal data is wirelessly communicable from the first device to the second device.

### RELATED APPLICATIONS

This application claims priority to copending U.S. Provisional application having Ser. No. 62/289,811 filed on Feb. 1, 2016; the contents of which are hereby incorporated by reference herein in their entirety.

### BACKGROUND

Field of the Invention

The present disclosure relates to a fetal monitoring systems and methods. More particularly, the present disclosure relates to a fetal monitoring system, fetal entertainment system, and related methods for (i) collecting and monitoring fetal data, and (ii) providing entertainment to a fetus.

Description of the Related Art

There are currently more than 6.7 million pregnancies per year in the United States and more than 200 million pregnancies per year worldwide. In the United States, fetal monitoring is performed at various stages during pregnancy, starting at around 6-8 weeks with an ultrasound. Fetal monitoring is continued throughout the pregnancy, and in certain countries, monthly monitoring is performed using, for example, ultrasound imaging. Ultrasound imaging can yield data, such as approximate fetal size and weight, organ development, placenta and umbilical cord structure and location, and fetal heart rate. Ultrasound imaging may be performed intravaginally or on the outside of the patient's stomach.

For higher risk patients, including both the mother and the fetus, fetal monitoring may be performed more frequently. Higher risk patients are subject to increased testing and monitoring, most of which require trips to the healthcare provider's office or hospital. Data is gathered; tests are preformed and sent to the healthcare provider or other party. Follow-up visits are scheduled to review the data and decide further directives. Improvements in the field of fetal monitoring are desired.

Further, a need exists to provide fetal monitoring data directly to the patient without the need to review with a healthcare provider. Current technology includes fetal heart rate monitoring devices in which a patient can listen to the fetal heart rate through a stethoscope. Improvements in the field of communicating fetal data directly from the fetus to the patient are desired.

### SUMMARY

In one embodiment of the disclosure, a method of monitoring a fetus in utero is described that includes implanting a medical device into a patient's uterus, collecting fetal data using the medical device, and transmitting the fetal data from the medical device to a receiver.

In certain embodiments, the method may further include securing the medical device within the patient's uterus, e.g., to an interior wall of the patient's uterus.

In certain embodiments, the method may further include positioning the medical device within an amniotic sac.

In certain embodiments, the method may further include navigating the medical device within the amniotic sac.

In certain embodiments, the method may further include securing the medical device to an exterior surface of the amniotic sac.

In certain embodiments, transmitting the fetal data may include wirelessly transmitting the fetal data to the receiver, e.g., to a location external of the patient's uterus.

In certain embodiments, wirelessly transmitting the fetal data may include wirelessly transmitting the fetal data to a receiver via a computer, a cloud, a robotic operating system, and/or a portable electronic device.

In certain embodiments, the method may further include inserting the receiver into the patient, and transmitting the fetal data may include transmitting the fetal data to the inserted receiver.

In certain embodiments, the method may further include removing the receiver from the patient.

In certain embodiments, the method may further include downloading the fetal data from the receiver.

In certain embodiments, collecting the fetal data may include capturing at least one image of the fetus, e.g., one or more still photographs of the fetus and/or video of the fetus, and transmitting the fetal data includes transmitting the at least one image to the receiver.

In certain embodiments, collecting the fetal data may include collecting data pertinent to health of the fetus, e.g., data selected from the group consisting of fluid levels, fluid compositions, fetal sounds, intra-uterine images, and electrical signals.

In certain embodiments, collecting the fetal data may include measuring or analyzing anatomical structures.

In certain embodiments, collecting the fetal data may include collecting a biological sample from the fetus, amniotic fluid, amniotic sac, placenta, or uterine wall.

In another aspect of the present disclosure, a medical monitoring system is disclosed that includes a first device that is implantable into a patient's uterus for collecting fetal data, and a second device that is configured and dimensioned for connection with the first device such that the fetal data is wirelessly communicable from the first device to the second device.

In certain embodiments, the first device may include hardware and software to facilitate collection of data, such as fetal images, fluid levels, fluid compositions, fetal sounds, intra-uterine images, and electrical signals.

In certain embodiments, the hardware and software of the first device may facilitate collection of images using ultrasound.

In certain embodiments, the hardware and software of the first device may facilitate collection of infrared signals.

In certain embodiments, the first device may include an anchoring structure to anchor the first device to a portion of the patient's uterus.

In certain embodiments, the first device may include navigation structure permitting controlled navigation of the first device within the patient's uterus from an external location.

In another aspect of the present disclosure, a method of monitoring a fetus in utero is disclosed that includes implanting a first device into a patient's uterus, collecting fetal data using the first device, removing the first device from the patient's uterus, and wirelessly connecting the first device to a second device such that the fetal data is wirelessly communicable from the first device to the second device.

In another aspect of the present disclosure, a system is disclosed for monitoring a fetus in utero that includes a first medical device implantable into a patient's amniotic sac for wirelessly collecting and transmitting fetal data, and a receiver for receiving the fetal data, wherein the receiver may include a computing cloud, a computer, a portable electronic device, and a second medical device.

In certain embodiments, the first medical device and the receiver may each include hardware and software facilitating wireless communication of the fetal data from the first medical device to the receiver.

In certain embodiments, the hardware and software of the first medical device and the receiver may facilitate wireless communication of data from the receiver to the first medical device.

In certain embodiments, the first medical device may include an anchoring structure to anchor the first medical device to a portion of the patient's amniotic sac.

In another aspect of the present disclosure, a method of monitoring a fetus in utero is disclosed that includes implanting a wireless medical device into a patient's uterus, and collecting fetal data using the medical device.

In certain embodiments, the method may further include securing the medical device within the patient's uterus, e.g., to an interior wall of the patient's uterus.

In certain embodiments, the method may further include positioning the medical device within an amniotic sac.

In certain embodiments, the method may further include navigating the medical device within the amniotic sac.

In certain embodiments, the method may further include securing the medical device to an exterior surface of an amniotic sac.

In certain embodiments, the method may further include transmitting the fetal data to a receiver external of the patient's uterus, e.g., wirelessly.

In certain embodiments, wirelessly transmitting the fetal data may include wirelessly transmitting the fetal data to a cellular phone.

In certain embodiments, collecting the fetal data may include capturing at least one image of the fetus, e.g., one or more still photographs and/or video of the fetus, and transmitting the fetal data includes transmitting the at least one image to the receiver.

In certain embodiments, collecting the fetal data may include collecting data pertinent to health of the fetus, e.g., data selected from the group consisting of fluid levels, fluid compositions, fetal sounds, intra-uterine images, and electrical signals.

In certain embodiments, collecting the fetal data may include measuring a body temperature of the fetus.

In certain embodiments, collecting the fetal data may include collecting a sample of blood and/or tissue from the fetus.

In another aspect of the present disclosure, a surgical kit is disclosed that includes: (i) a first medical device configured and dimensioned for implantation into a patient's uterus that includes hardware and software facilitating collection of data from within the patient's uterus, as well as transmission of the data to a location exterior of the patient's uterus; and (ii) a receiver that is configured and dimensioned for connection with the first device such that the data is wirelessly communicable from the first device to the second device.

In certain embodiments, the hardware and software of the first medical device may facilitate collection of the data, such as fetal images, fluid levels, fluid compositions, fetal sounds, intra-uterine images, electrical signals, and infrared signals.

In certain embodiments, the hardware and software of the first medical device may facilitate collection of the intra-uterine images using ultrasound.

In certain embodiments, the first medical device may include navigation structure permitting controlled navigation of the first medical device within the patient's uterus.

In certain embodiments, the surgical kit may further include a second medical device configured and dimensioned for implantation into the patient's uterus that includes hardware and software facilitating collection of data from within the patient's uterus, and transmission of the data to the location exterior of the patient's uterus.

In certain embodiments, the first and second medical devices may be programmable to collect different types of data.

In certain embodiments, the first medical device may be programmable to collect data pertinent to health of the fetus, and the second medical device may be programmable to collect data pertinent to health of the patient.

In certain embodiments, the first medical device may be programmable to collect information regarding the heart rate of the fetus, and the second medical device may be programmable to collect information regarding the heart rate of the patient.

In certain embodiments, the first medical device may be programmable to collect images of the fetus, and the second medical device may be programmable to collect images of the patient's uterus.

In certain embodiments, the first and second medical devices may each include an anchoring structure to anchor the first and second medical devices in relation to the patient's uterus.

In certain embodiments, one of the first and second medical devices may be configured and dimensioned for implantation into an interior wall of the patient's uterus, and the other of the first and second medical devices may be configured and dimensioned for implantation into an exterior wall of the patient's uterus.

In certain embodiments, the first and second medical devices may each be configured and dimensioned for implantation into an interior wall of the patient's uterus.

In certain embodiments, at least one of the first and second medical devices may be configured and dimensioned for implantation into an amniotic sac.

In certain embodiments, the first and second medical devices may each be configured and dimensioned for implantation into the amniotic sac.

In certain embodiments, a method for communicating with a fetus may include providing, on an electronic device, a user interface configured to facilitate wireless communication of signals between the electronic device and at least one implanted device located in proximity to the fetus; and responsive to a user selecting a communication selection element, causing the wireless communication of signals inclusive of content data between the electronic device and implanted device.

In certain embodiments, causing the wireless communication of signals may include communicating image data of the fetus captured by the implanted device to the electronic device.

In certain embodiments, causing the wireless communication of signals may include communicating video data captured by the implanted device to the electronic device.

In certain embodiments, causing the wireless communication of signals may include communicating audio data from the fetus captured by the implanted device to the electronic device.

In certain embodiments, causing the wireless communication of signals may include communicating audio data from the electronic device to the implanted device for an audible output by the implanted device.

In certain embodiments, a selection element may be provided on the user interface that enables the user to select music from a selectable list of music; and responsive to the selection of music, may cause the music to be communicated from the electronic device to the implanted device.

In certain embodiments, providing a selection element on the user interface may enable the user to select a video chat user interface; and causing a 2-way communication that may enable the electronic device to receive video data from the implanted electronic device and to communicate audio data from the user via the electronic device to the implanted electronic device.

In certain embodiments the method may include, communicating video data captured by first and second implanted devices to the electronic device; processing the video data received from the first and second implanted electronic devices to produce 3D video data; and displaying the 3D video data on the electronic device.

In certain embodiments, the method may include providing a graphical user element that enables a user to enter information and select pre-established graphics; and displaying the entered and/or selected information or selected pre-established graphics on an electronic display of the electronic device in relation to an image of the fetus from content data received from the implanted device.

In certain embodiments, displaying the information or graphics may include displaying text and/or graphical images.

In certain embodiments, the method may include establishing preset messages; and responsive to determining that a signal received from the implanted device corresponds to a preset message, causing the preset message to be displayed on an electronic display of the electronic device.

In certain embodiments, a fetus entertainment system, includes at least one implantable device configured and adapted to capture data representative of an image of a fetus and to produce audio signals for transmission to the fetus; and user interface software configured to be executed on an electronic device, and provide a user with a graphical user interface configured to: enable a user to cause the electronic device to communicate wireless signals to, and receive wireless signals from, the at least one implantable device, while implanted in proximity to the fetus; and present a user selection element that, when selected by the user, causes the electronic device to communicate the wireless signals to the at least one implantable device.

In certain embodiments, the user interface may include a selection element that enables the user to select music from a selectable list of music for wireless communication to the at least one implantable device.

In certain embodiments, the system may include wherein the at least one implantable device is configured, dimensioned, and adapted to capture images and/or video of the fetus and communicate captured image and/or video data to the electronic device, and wherein the user interface software facilitates receipt of the image and/or video data communicated from the at least one implantable device.

In certain embodiments, the user interface may include a selection element that enables the user to select a video chat user interface facilitating 2-way wireless communication between the electronic device and the at least one implantable device such that the electronic device receives video data from the at least one implantable electronic device, and the electronic device communicates audio data from the user to the at least one implantable electronic device.

In certain embodiments, the at least one implantable device may include first and second implantable devices, the first and second implantable devices each being configured and adapted to capture and communicate video data to the electronic device, the user interface software processing the video data received from the first and second implantable devices to produce 3D video data for display on the electronic device.

In certain embodiments, the user interface software may include a graphical user element that enables a user to enter information and select pre-established graphics, the entered and/or selected information or selected pre-established graphics being displayable on the electronic device in relation to an image of the fetus generated from data received from the at least one implantable device.

In certain embodiments, the user interface software may include preset messages for display on the electronic device in response to determining that a signal received from the at least one implantable device corresponds to one of the preset messages.

In certain embodiments, a method for communicating with a fetal environment, includes providing, on an electronic device, a user interface configured to facilitate communication of signals between the electronic device and at least one implanted device located in proximity to a fetus; and responsive to a user selecting a communication selection element, causing the communication of signals inclusive of content data between the electronic device and implanted device.

### BRIEF DESCRIPTION OF THE DRAWINGS

These and other features, aspects and advantages are described below with reference to the drawings, which are intended to illustrate but not to limit the inventions. In the drawings, like reference characters denote corresponding features consistently throughout similar embodiments. The following is a brief description of each of the drawings.

FIG. 1 is a schematic view of an expectant patient together with a medical device in connection with a receiver according to the present disclosure;

FIG. 2 is a schematic view illustrating one embodiment of the presently disclosed medical device

FIG. 3 is a schematic view illustrating an alternate embodiment of the presently disclosed medical device;

FIG. 4 is a schematic view illustrating an alternate embodiment of the presently disclosed medical device;

FIG. 5 illustrates a system in which a pair of medical devices are utilized;

FIG. 6 is a schematic view illustrating an alternate embodiment of the presently disclosed medical device;

FIG. 7 illustrates a sensor for use with various embodiments of the presently disclosed medical device;

FIG. 8 is a schematic view illustrating an alternate embodiment of the presently disclosed medical device;

FIG. 9 is a schematic view illustrating an alternate embodiment of the presently disclosed medical device;

FIGS. 10-15 illustrate various methods of using the presently disclosed medical devices to collect and/or transmit data;

FIG. 16 is a screenshot of an illustrative electronic device executing an illustrative user interface for communicating with and controlling a medical device, as previously described;

FIG. 17 is a flow diagram of an illustrative process for communicating with a fetus;

FIG. 18 is a block diagram of an illustrative electronic device configured to support functionality for communicating with a fetus as described herein; and

FIG. 19, a set of engines that may be executed by the processing unit.

### DETAILED DESCRIPTION

While the present description sets forth specific details of various embodiments, it will be appreciated that the description is illustrative only and should not be construed in any way as limiting. Furthermore, various applications of such embodiments and modifications thereto, which may occur to those who are skilled in the art, are also encompassed by the general concepts described herein. Each and every feature described herein, and each and every combination of two or more of such features, is included within the scope of the present invention provided that the features included in such a combination are not mutually inconsistent.

With respect to the terminology used herein, it should be noted that the term “fetus” or “fetal” as used herein or includes an embryo of less than about 10 weeks of age to a full-term fetus of about 40 weeks or until a birth event. Additionally, the terms “collect,” “capture,” “gather,” “receive,” and variations thereof may be used interchangeably throughout the present disclosure in the context of acquiring data. The term “data” as used herein may include information, images, videos, audio, biological samples, and other sensed information. The data may be communicated wirelessly, and be in any format and utilize any protocol.

Pregnant women are routinely monitored to monitor health of a fetus. Ultrasound is generally used to measure certain parameters of a fetus to ensure proper development and health. In cases where certain abnormalities are measured, additional ultrasound and other sensing may be performed to more closely monitor the fetus. In these cases, the pregnant woman is to visit health facilities, where a trained technician and/or medical professional is to use an ultrasound system to examine the fetus. These in-office visits, which beneficial to the mother, fetus, and medical professional, may be also time and labor intensive, along with financially burdensome for the pregnant woman and insurance company.

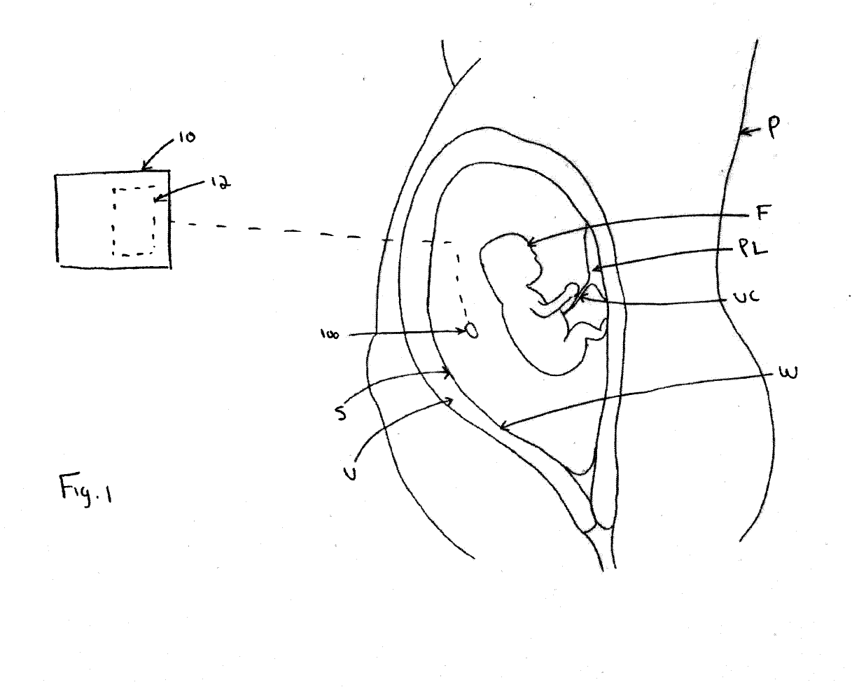

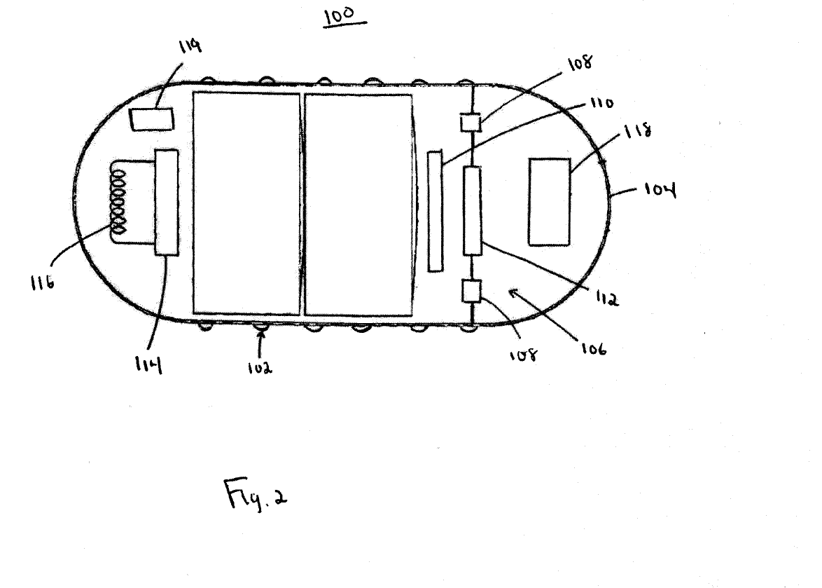

FIGS. 1 and 2 illustrate an expectant patient P having a developing fetus F within her uterus U, as well as a medical device 100 (described hereinbelow) for use in collecting data pertaining to the patient P and/or the fetus F. With specific reference to FIG. 1, the fetus F is positioned within an amniotic sac S located within the patient P's uterus U, and is connected by an umbilical cord UC to a placenta PL. It should be understood that the anatomical organs and structures described herein function as normal organs and structures during fetal development.

### Medical Device Components

In particular, fetal monitoring systems of the present disclosure include at least one medical device 100 configured for implantation within a patient P (FIG. 1). In general, upon implantation, the medical device can collect or gather fetal data and transmit the fetal data to various components of the fetal monitoring system including at least one receiver. Optionally, upon receipt of the fetal data, a medical or healthcare directive may be decided.

Medical device 100 of the present disclosure may include hardware and software components for collecting and transmitting fetal data (described hereinbelow). In embodiments, the medical device 100 may include one or more of transmitters/transmission systems, power source (e.g., battery), antenna, control system, positioning device(s), sensor(s) (e.g., imaging, acoustic, thermal, etc.), navigation means, imaging systems, and so forth. Each of the system components may be sized to be positioned/disposed within the medical device 100. To facilitate the collection of fetal data, in one embodiment of the disclosure, the medical device 100 may include hardware and software that support an imaging system 106 including an optical window 104 for obtaining images from inside the uterus U, e.g., video and/or photographs (FIG. 2). For example, the imaging system 106 may include a source 108 of illumination, such as an LED (light-emitting diode), to provide illumination through the optical window 104, as well as an image detector 110, such as a camera, and an optical system 112 (e.g., lens or set of lenses), which focuses the images onto the image detector 110. The imaging system can collect fetal data including but not limited to images and recordings of the fetus, and images and recordings of patient anatomy within the uterus. The imaging system 106 may work together with a transmitter (later described) to transmit fetal and patient data (e.g., images, recordings) to a receiver 10 (also later described). In particular, the imaging system 106 can may communicate with a data processing unit 12 (FIG. 1) of the receiver 10 in any suitable manner, e.g., via a small flexible wire or cable, or using wireless technology (see FIG. 2).

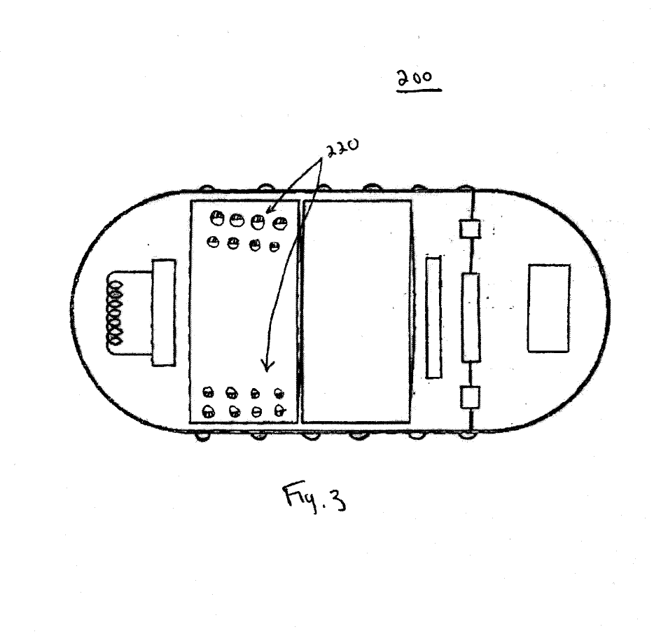

In another embodiment, the medical device 200 of FIG. 3 may include hardware and associated software that facilitates the capture and transmission of ultrasound data. For example, the medical device 200 may include one or more ultrasound transducers 220, e.g., in the form of a linear array, a phased array, a sector array, or a multi-dimensional array, in communication with a transmission system to generate two-dimensional and/or three-dimensional images. It is envisioned that the transducer(s) 220 may communicate with the data processing unit 12 (FIG. 1) of the receiver 10 in any suitable manner, e.g., via a small flexible wire or cable, or using wireless technology. Although not fully shown, it is contemplated that components and functionality of the medical device 200 may be the same or similar as those shown in FIG. 2.

Associated frequency ranges are within the purview of those skilled in the art and include, but are not limited, to 5 to 100 Megahertz. One example of a suitable ultrasound transducer 220 is described in U.S. Patient Publication No. 2001/0031924, assigned to Mayo Foundation for Medical Education and Research, the entire content of which is incorporated by reference herein.

The ultrasound data captured and transmitted by the medical device 200 may include sonographic images and/or recordings, e.g., 2-Dimensional (2D), 3-Dimensional (3D), 4-Dimensional (4D) data, or continuous-wave (CW) Doppler. 2D, 3D, and 4D data may include, or enable a healthcare provider to ascertain certain information pertaining to physical and/or anatomical characteristics of the patient P and/or the fetus F, e.g., the size and body structure of the fetus F (gender, bone length, fundal height, heart chamber size, head circumference, etc.), fetal growth, fetal movement, fluid levels, etc. In certain instances, CW Doppler, which differs from traditional sonographic techniques in that it does not require the generation of an image, may be utilized where a change in frequency and phase of reflected ultrasound signal is measured. For example, CW Doppler may be utilized to measure fluid flow in the umbilical cord, fetal blood vessels, veins, arteries, etc.

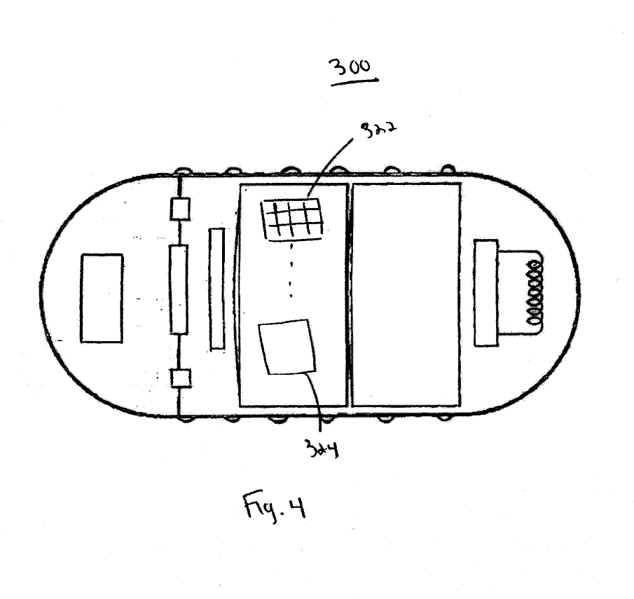

Medical devices according to the present disclosure may also include thermal or temperature sensing capabilities, e.g., to measure and/or record the temperature of the amniotic fluid, the temperature of the patient P, the temperature of the fetus F, etc. For example, in one embodiment, a medical device 300 (FIG. 4) is disclosed that includes a thermal sensing system having an image sensing module 322 in communication with an integrating unit 324, e.g., to detect the dark current of an image, and may amplify or otherwise process the data, if necessary, to calculate environmental temperature based on known equations derived from thermal noise. Alternatively, temperature may be calculated by a separate data processing unit that is capable of integrating thermal noise and determining temperature. Thermal sensing system may communicate with a receiver in any suitable manner, e.g., via a small flexible wire or cable, or using wireless technology.

It is also envisioned that temperature, e.g., the temperature of the fetus F, may be calculated using know factors, such as heat distribution, distance from the image sensing module 324, etc. Suitable devices and corresponding methodologies for collecting thermal noise data are described in U.S. Pat. No. 6,607,301, assigned to Given Imaging Ltd., the entire content of which is incorporated by reference herein.

In certain embodiments, the medical devices 100, 200, and 300, may include structures facilitating controlled movement or navigation within the patient P. For example, the medical device 100 may include a controllable drive system and/or a positioning system in communication with an external source to remotely control movement and positioning of the medical device 100 within the patient P. For example, it is envisioned that the drive system may include a motor or may be magnetically controlled. In embodiments, the drive system may be wirelessly controlled from outside or exterior to the patient's body. The drive system may be configured with a propeller, screw drive, or other fluid propulsion system to travel within fluid or feet or other members that are configured to move the medical device 100 along tissue. In one embodiment, an anchor mechanism (not shown), such as one or more screws or other tissue attachment member(s) (e.g., barbs or projections disposed on an exterior surface of medical device), may enable the medical device to self-position itself in a fixed position.

Alternatively, magnetically controlled drive systems may include in particular a magnet disposed within the medical device. A complementary, oppositely charged magnet or electromagnet may be placed exterior to the patient's uterus and be moved in specific directions to drive or influence the movement of the medical device. In this embodiment, opposite or similar magnetic charges can drive the movement of the magnet within the medical device, and, in turn, drive the movement of the medical device. It should be noted that magnets should be strong enough to drive movement of the medical device, but not so strong as to damage any interior patient tissue. The movement may be positioning of the device in the uterus, orientation, or component movement (e.g., camera angle). In an alternative embodiment, the medical device may not include a drive system, but may rather be surgically implanted and internal control device(s) may be utilized to reposition a sensor (e.g., camera) within the medical device 100.

As indicated above, the various embodiments of the medical devices described herein, e.g., the medical device 100 (FIGS. 1, 2), are each configured, dimensioned, and adapted to detect, collect, record, and/or transmit, e.g., wirelessly, various types of data pertaining to the patient P and/or the fetus F. In various embodiments, the medical device 100 may be adapted to detect, collect, record, and or transmit information pertaining to the health of the fetus F and/or the patient P, e.g., fluid levels, fluid compositions, fluid flow (e.g., blood flow), heart rate of the patient P and/or the fetus F, the body temperature of the patient P and/or the fetus F, hormone levels, brainwave data, or other anatomical characteristics (e.g., size measurements), fetal sounds, intra-uterine images, electrical signals, infrared signals, ultrasound imaging, electroencephalogram (EEG) data, electrocardiogram (ECG or EKG) data, visual data, such as photographs or video, and/or tissue specific (optical) data, as described in U.S. Patent Application Serial No. 2014/0228653, the entire content of which is incorporated by reference herein. Although it is envisioned that the various embodiments of the medical devices described herein may include hardware and software to support the detection, collection, recordation, and/or transmission of multiple types of data, it is also envisioned that a medical device according to the principles of the present disclosure may include hardware and software to support the detection, collection, recordation, and/or transmission of a single type of data. In one embodiment, the medical device, e.g., the medical device 100 (FIGS. 1, 2), may be include one or more sensing devices (e.g., microphone, audio transducer, ultrasound transducer, pressure sensor, temperature sensor, and/or other sensor) to sample one or more signals, such as audio and/or ultrasound signals, and communicate raw data signals to a remote receiver of a system (e.g., mobile device) configured to receive and process the signal(s) captured by the medical device 100.

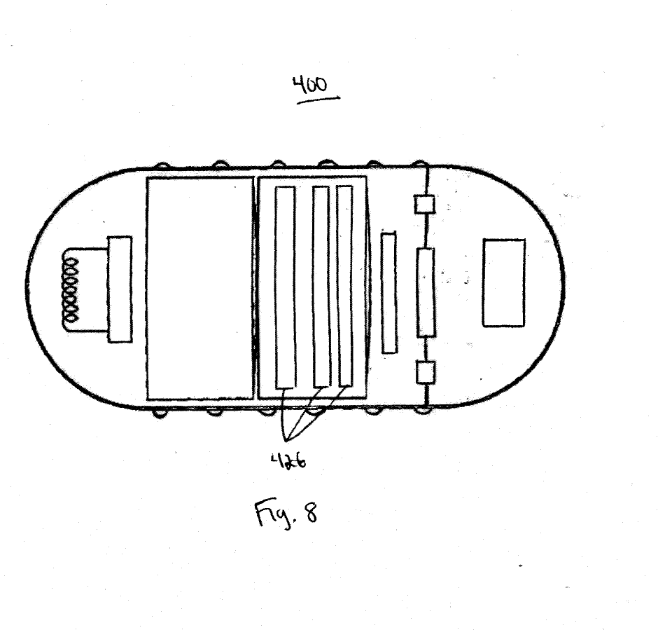

Because the medical device, e.g., the medical device 100, may be implanted with a region subject to movement, e.g., by the patient P and/or by the fetus F, the medical device 100 may be configured with a sensor, such as an ultrasound transducer, mounted to a substrate supported by a gimbal mount that enables the transducer to be rotated in one or two axes, as illustrated in FIG. 6. The gimbal mount may include an electromechanical device that allows for the sensor to be repositioned to point at a region of interest, such as a heart of the fetus. In one embodiment, the gimbal mount may include, or be configured as, a micro electromechanical system (MEMS). In an alternative embodiment, rather than using a gimbal mount, a phased array of sensors may be utilized, such as a phased array of antenna elements, that enables a beam to be directed as a function of a phased signal, as understood in the art, that is input into the phased array of antenna elements. In yet another embodiment, an electromechanical or electronic zoom of a camera may be utilized, where the electronic zoom may have no moving parts, but rather uses an image sensor having sufficient number of pixels to enable an image to be digitally zoomed to a certain region-of-interest captured within a field-of-view of a camera or other sensor type.

In those embodiments utilizing a gimbal mount, a sensor may be automatically repositioned to sense a desired region-of-interest or audio source. In automatically repositioning the sensor, the sensor may be repositioned so that an audio, image, and/or thermal signal is at a maximum level by using a search pattern. The search pattern may follow a predetermined search path, such as a raster scan, and identify local maximums throughout the search path and identify the maximum of the maximums. Alternatively, the sensor may be dynamically adjusted to continuously follow a maximum level, such that if the sensor finds a local maximum and searches around the local maximum in a circular, square, or other pattern to determine if higher levels may be identified.

In one embodiment, a sensor may be configured with quadrants, as illustrated in FIG. 7, and may be repositioned as a target signal is determined by performing a difference between a sum of signals captured along the diagonals of the sensor, as understood in the art. For example, if a determination is made that a signal is strongest in an upper left quadrant, the sensor may be rotated up and left (a more defined angle may be determined using signal processing). In an alternative embodiment, rather than being automatic, a manual process may be utilized via a receiver 10 by enabling a user of the receiver 10 to communicate a command to move the sensor via the electronically controlled gimbal. The signal being sought may have a certain image, audible (e.g., heartbeat), electrical signature, chemical signature, or any other signature that may be processed using signal and/or image processing that may be used to guide or provide feedback to control movement or orientation of the sensor or component (e.g., gimbal).

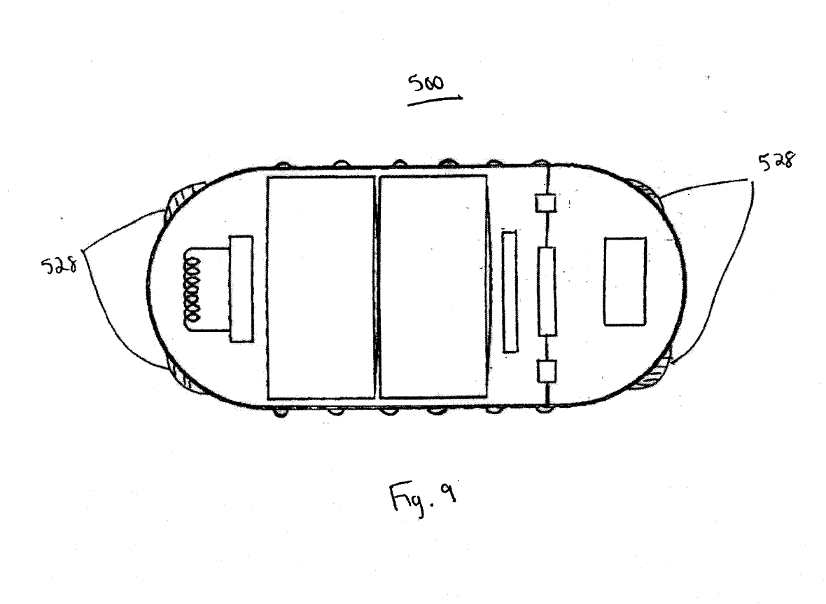

With reference now to FIG. 8, another embodiment of the medical device, identified generally by the reference character 400, will be discussed which includes one or more reservoirs 426, e.g., one or more pockets, channels, or other such cavities, to collect material capable of providing fetal data, e.g., bodily fluid samples, tissue samples, etc., for analysis. For example, the reservoir(s) 426 may retain the material collected, and thus, may serve as a vessel or vehicle (mechanism) for transporting the collected material, to a location exterior to the patient P, e.g., to a laboratory or hospital. Alternatively, the material collected by the reservoir(s) 426 may be analyzed in-situ by hardware provided within the medical device, as described in U.S. Pat. No. 8,370,068, the entire content of which is incorporated by reference herein.

Additionally, or alternatively, the reservoir(s) 426 may be configured, dimensioned, and adapted to dispense one or more agents, e.g., biopharmaceuticals, pharmaceuticals, physiological treatments, bioactive agents, etc., which may have therapeutic properties, and each of which may be sized accordingly, e.g., of micro or nano-scale. In certain embodiments, it is envisioned that multiple agents including more than one type or configuration of an agent may be preloaded into the reservoir(s) 426 and dispensed or released in situ. Contents of therapeutic reservoirs may be provided or configured before the sensor is implanted in or attached to organism, or may be manufactured and filled in vivo by a therapeutic manufacturer/provider. Therapeutic reservoirs may release or dispense contents when appropriately signaled by transmitting or processing unit. Suitable reservoir systems include but are not limited to U.S. Pat. No. 8,370,068, assigned to Dennis Fernandez, the entire content of which is incorporated by reference herein.

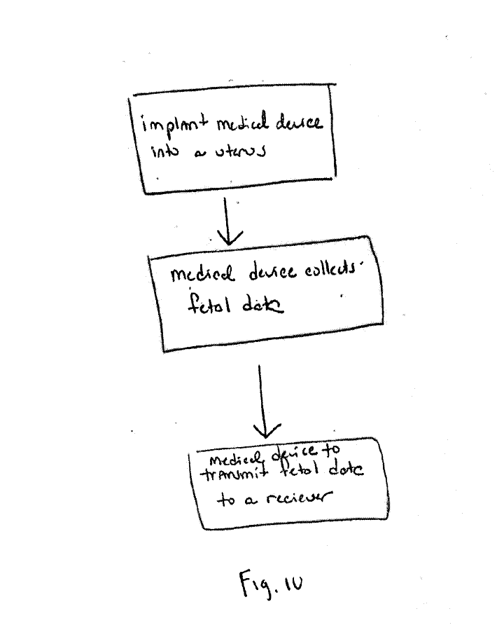

With reference to FIG. 9, in another embodiment, a medical device identified generally by the reference character 500 is disclosed that includes one or more biosensors 528 to provide fetal data related to the surrounding environment. As illustrated in FIG. 9, for example, the biosensor(s) 528 may be provided on an exterior of the medical device 500 such that the biosensor(s) 528 may interact with the surrounding tissues, fluids, etc. in gathering data. In certain embodiments, the biosensor(s) 528 may be configured, dimensioned, and adapted to detect and/or quantify chemical data, e.g., chemical compositions, pH levels, hormone levels, etc. In certain embodiments, the biosensor(s) 528 may include a material reactive with fluids to optically display pH levels, e.g., as an indicator of fetal kidney function.

The biosensor(s) 528 may be configured, dimensioned, and adapted to relay data upon removal of the medical device 100 from the patient P. It is envisioned that the biosensor(s) 528 may include a material that swells, absorbs, or otherwise collect material, e.g., fluid from the surrounding environment, e.g., the amniotic sac S, such that the material may be removed with the medical device 100. For example, the biosensor(s) 528 may include a swellable polymer, e.g., a hydrogel, which may collect fluid, e.g., amniotic fluid, uterine tissue, and the like. Following removal of the medical device 100, and thus, the biosensor(s) 528, a healthcare provider may directly test the material collected by the biosensor(s) 528, e.g., by extracting chemical compositional information, hormone levels, etc., using know methods.

In other embodiments, the biosensor(s) 528 may be optical sensors used for detection and quantification of chemical compositions, pH levels, hormone levels via analytes found within the fetal environment. Suitable optical sensors, include, for example U.S. Pat. No. 6,625,479, assigned to Torsana Diabetes Diagnostics A/S, the entire content of which is incorporated by reference herein. Additional information, and suitable examples of employable receivers 10, data processing units 12, etc., are described in U.S. Pat. No. 8,290,556, the entire content of which is incorporated by reference herein.

With reference again to FIGS. 1 and 2, as briefly described, the transmitter 114 may be configured to transmit collected data to the receiver 10, which includes a data processing unit 12, where the collected data may be processed, e.g., fed into mathematical algorithms, equations and the like, re-configured, altered, changed, or otherwise manipulated for presentation in an interpretable format. The transmitter 114 may be configured to communicate in a variety of frequency ranges utilizing analog or digital communications, as understood in the art. An antenna 116 of the transmitter 114 may be configured with an omnidirectional antenna pattern so that orientation of the medical device 100 does not impact communications. Alternative antenna patterns may be utilized. In one embodiment, the antenna pattern may be directional such that wireless signals are communicated away from the fetus to avoid radiating the fetus. Signal strength from the transmitter 114 via the antenna may be set at a level that complies with FCC regulations given that the medical device will be used in relation to a human (and fetus). Conventional or proprietary communications protocols may be utilized for the wireless or wired communications. In certain embodiments, it is envisioned that the data processing unit 12 may be separate from the receiver 10, and may act to communicate or transmit collected data between the medical device 100 and the receiver 10.

The transmitter 114, which may be part of a transmitter system inclusive of a number of other communications components, such as the antenna 116, may be used to transmit data representative of images collected by the image detector 110. A power source 118, e.g., a battery, may be utilized to provide power to the electrical elements of the medical device 100. It is envisioned that the power source 118 may be rechargeable, non-rechargeable, replaceable, or may operate by direct connection or power scavenging, such as an inductively charged capacitor. In those embodiments including a rechargeable power source 118, the medical device 100 may include a transformer (not shown) that enables inductive coupling with another inductor that may be remotely positioned, e.g., outside of the patient P, to transfer power to the medical device 100 to charge the power source 118. It should be understood that a power source may be utilized to power any of the embodiments described above.

It is further envisioned that the power source 118 may provide power to the medical device 100, or only to a portion thereof. In alternate embodiments of the disclosure, the number and types of power sources 118 may be varied to power the various electrical elements of the medical device 100 without departing from the scope of the present disclosure. Alternative techniques may also be utilized to power the rechargeable device once implanted within a mother/patient. One example of a medical device including an imaging system is described in U.S. Pat. No. 8,681,201, assigned to Given Imaging Ltd, the entire content of which is incorporated by reference herein. To facilitate the collection and/or analysis of data, it is envisioned that the medical device 100 may include a processor 119, as illustrated in FIG. 2 for example.

The medical device 100, in certain embodiments, may further include a receiver (not illustrated) disposed therein/thereon that is configured to receive command or control signals to control operation of the medical device 100. The receiver may be an electronic circuit that utilizes a communications protocol that is conventional, such as Bluetooth, WiFi, or any other conventional or proprietary communications protocol. The commands may be used to control motion, start and stop sensing, change mode, set parameters, turn on/off, or cause one or more functions of the medical device 100 to turn on, turn off, or otherwise be altered.

The medical device 100, 200, 300, or otherwise, and the various embodiments thereof described herein, may be configured and dimensioned for implantation into the patient P's uterus, either partially or entirely, on either a permanent or temporary basis. For example, the medical device 100 may be implanted into, or otherwise secured to, the patient P's uterine wall W (FIG. 1), the amniotic sac S, or to any other suitable internal structure, organ, or the like. To facilitate such implantation, the medical device 100 may include anchoring structure(s) 102 (FIG. 2), which may be mechanical in form, e.g., hooks, barbs, eyelets, clips, sutures, staples, or the like, and/or chemical in form. In more detail, mechanical means, such as hooks, barbs, spikes, or teeth, may be disposed on an exterior surface of the medical device 100, and may be configured to facilitate attachment to patient's tissue. Other structures, similar to barbed sutures, or Velcro are also envisioned. In another non-limiting example, an eyelet may be formed monolithically with medical device 100 and the medical device 100 and a suture may be passed through the eyelet to anchor the medical device 100 to the patient's tissue. Alternatively, sutures, staples, or clips may be attached to or provided with the medical device 100 to facilitate attachment of the medical device to the patient. The design of sutures, staples and clips may be modified in shape and/or size to facilitate attachment of the medical device 100. In yet other embodiments, the anchoring structure(s) 102 may include an adhesive, or other suitable chemical compositions, facilitating temporary or permanent attachment to tissue, such as, for example, peptides, isocyanates, cyanoactylates, and the like.

In one particular embodiment, the medical device 100 may be configured as a capsule or pill including smooth edges and peripheral geometry, as seen in FIGS. 1 and 2, for example, so as to not tear, damage or disrupt the surrounding environment, e.g., the patient P's uterus U. In other embodiments, however, the medical device 100 may be configured as a patch, mesh, tape, strip, or buttress, for example, without departing from the scope of the present disclosure.

Although shown in different embodiments, the various sensing devices in the different medical devices 100, 200, and 300 may be combined into a medical device, e.g., a single capsule, which may be enlarged to support additional sensing components. If multiple sensing devices are utilized in a single medical device, then each of the sensing components may be operated in combination or independently. For example, a medical device may be configured to have a camera turned on in conjunction with a thermal imager, and, in one embodiment, the camera may be controlled to be positioned toward a highest thermal temperature. Other types of controls that utilize different sensors in a single medical device may be utilized, as well.

It should be understood that medical devices of the present disclosure can include one or more of the data acquisition systems, drive or navigation means and/or anchoring structures.

### Various System Components

In more detail, the medical device 100 may be in communication with a receiver 10, e.g., a portable computer system, such as a mobile/smart phone, etc., for the receipt, storage, and/or processing of data collected by the medical device 100. As elaborated upon in further detail herein, the medical device 100 collects and/or transmits fetal and/or patient data to the receiver 10, via either wireless or physical communication.

In particular, the receiver 10 may encompass, or be executable by, portable and non-portable electronic devices and technologies. In one embodiment, the receiver 10, such as a mobile phone, may include computing and Internet access capabilities, such as those capabilities provided by cellular and/or smart phones, personal device assistants (PDA), etc. It is further envisioned that the receiver 10 may encompass or be included as part of an electronic devices, such as desktop computers, portable computers, laptops, smart tablets, e.g., an iPad, smart televisions, MP3 players, smart eyewear etc.

It is further envisioned that the receiver 10 may be configured and adapted to communicate with or be integrated with technologies having visualization capabilities, such as, for example, smart eyewear, e.g., Google Glass, smart contact lenses, and smart headsets. Alternatively, the receiver may be disposed within the smart eyewear.

Alternatively, the receiver 10 may be positioned in contact with an exterior portion of the patient P, a surgeon, etc., e.g., in the form of a wearable article, such as, for example, clothing, jewelry, glasses, headphones, a wristband, a headband, a bracelet, a watch, shoes, ear pieces, etc.

In various embodiments, the receiver 10 may either include, or be capable of interfacing with, cloud storage and computing systems, which should be understood as encompassing all manners of computing in which a larger group of remote servers are networked to allow centralized data storage and online access to computing services or resources. In such embodiments, collected data may be saved to a cloud for subsequent access by the patient P, a doctor, healthcare provider or another party, individual or corporation. Alternatively, the receiver 10 may either include, or be capable of interfacing with electronic healthcare/patient records.

In various embodiments of the disclosure, it is envisioned that the medical device 100 and the receiver 10 may each include hardware and software, such as a data processing unit 12, facilitating either uni-directional communication, e.g., from the medical device 100 to the receiver 10, or bi-directional communication (e.g. from the medical device 100 to the receiver 10 and from the receiver 10 to the medical device). It should be noted that more than one receiver 10 or medical device 100 can provide uni-directional or bi-directional communication. The data processing unit 12 may include one or more processors, such as general processors, digital signal processors, image processors, communications chips or processors, or any other processors that may provide for sensing, control, and/or communications algorithms to be executed thereon. It is envisioned that the receiver 10 may be located externally of the patient P, as illustrated in FIG. 1, or alternatively, that the receiver 10 may be located within the patient P, e.g., in the form of a separate medical device. In certain embodiments, the data processing unit 12 may be a component of the receiver 10. Alternatively, the data processing unit 12 and the receiver 10 may be separate, in which case, the data processing unit 12 and the receiver 10 may communicate in any suitable manner.

The receiver 10 may be wired or wireless, and may operate at any frequency and utilize any communications protocol capable of communicating data collected by the medical device 100. In one embodiment, a Bluetooth communications protocol may be utilized so that conventional mobile devices, such as smart phones, may be utilized to receive, process, and display data collected by the medical device 100, and optionally processed by the mobile device. Data processing of the collected data may be performed by the medical device 100 and/or the data processing unit 12 operating in conjunction with the receiver 10. The processing unit 12 may include signal processing, such as audio and/or image processing, to identify signals and improve signal-to-noise ratios, as understood in the art. In one embodiment, the signal processing may include filtering in order to reduce noise such that a signal-to-noise ratio is increased, as understood in the art. The specific filter(s) utilized may depend on the type of signal, type of noise, amount of processing power, and other factors. As an example, a filter that reduces noise created by a digestive tract, fluid movement, etc. may be utilized. In one embodiment, the filter may be a matched filter that is configured and adapted to identify a heartbeat of the patient P and/or the fetus F. The data processing unit 12 may also be configured and adapted to generate and display output data to be output from a speaker and/or displayed on an electronic display located on, or associated with, the receiver 12. Data files, such as audio, video, and/or image data files may be generated and stored in a data repository for later use.

The receiver 10 may also include, or be capable of interfacing with, augmented reality systems, as described in U.S. Patient Publication No. 2013/0125027, assigned to Magic Leap, Inc., the entire content of which is incorporated by reference. In such embodiments, the augmented realty system may provide a virtual 3D image of the fetus F in utero which may be projected or displayed, for example, in real time, e.g., for use in surgical planning. In another example, the augmented realty system may be used to create a virtual 3D image of the fetus post-surgery. Various examples of suitable augmented reality software and hardware is made available by Qualcomm, Vuforia, METAIO's SDK, Total Immersion, Sphero, POPAR, Sony, Nintendo, Microsoft, Layar, Aurasma, DAQUI, and Zappar.

As previously described, receivers may also be located within the medical devices in embodiments when bi-directional communication is required.

In those embodiments where the receiver 10 is provided as a second device or medical instrument or medical tool, the second device may be configured as a robotic operating system, tool, instrument, etc. For example, a robotic operating system, e.g., the minimally invasive robotic surgical system da Vinci®, may include a processing unit in communication with the first implantable medical device, e.g., the medical device 100 (FIGS. 1, 2). As indicated above, the receiver 10 (FIG. 1) may be positioned within the patient P's body, e.g., in the form of a second device, a medical tool, a medical instrument, a piece of equipment that is configured, dimensioned, and adapted to collect and/or relay data, for relaying data, which may be either temporarily or permanently placed. Examples include, but are not limited to, robotic tools and instruments, access tools and devices such as access ports, NOTES (natural orifice translumenal endoscopic surgery) instruments, SILS (single incision laparoscopic surgery) instruments, EndoWrist® instruments, each of which may contain the processing unit and a transceiver, for example, for receiving and/or transmitting data using any communications protocol capable of supporting communication of the data and/or other data derived therefrom.

Alternatively, the receiver 10 may be provided in the form of an ultrasound instrument, a CT instrument, and/or an MRI instrument. For example, MR imaging data may be collected in utero using the medical device 100, and may be then transmitted to a separate MRI instrument (having a processing unit) at a physician's office, where the data may be overlaid, combined or processed to create a more complete 4D or 5D image.

### Multiple Fetal Monitoring Systems

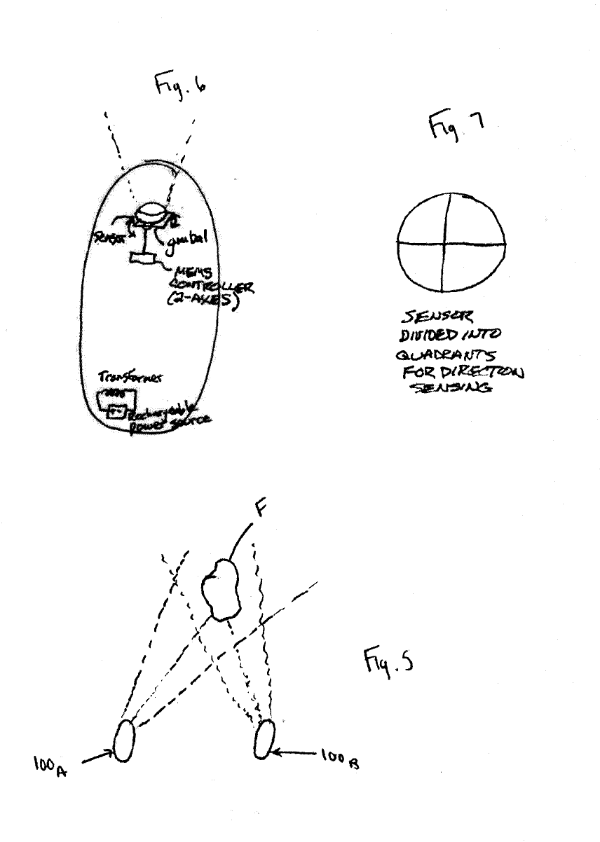

In one embodiment, illustrated in FIG. 5, for example, rather than using a single medical device 100, multiple medical devices 100_{A}, 100_{B} may be utilized and spaced at a distance that enables the medical devices 100_{A}, 100_{B} to capture signals in a stereoscopic manner or otherwise. By using multiple medical devices 100_{A}, 100_{B}, both two dimensional (2D) and three dimensional (3D) images may be captured. A 2D image provides for visual inspection in a planar manner, while a 3D image provides for images in a spatial manner with depth. The 3D image may be in the form of a depth map having a gray scale, where a close object is bright and a far object is dark. The depth map may be calibrated based on anticipated minimum and maximum ranges of images being captured. As understood in the art, to capture a 3D image, a distance between the medical devices 100_{A}, 100_{B}, and more specifically, sensors (not shown) associated with the medical devices 100_{A}, 100_{B}, is to be known. Because the distance between the sensors may vary, e.g., due to the location of the medical devices 100_{A}, 100_{B} within the patient P, calibration may be made to determine the distance of the sensors on a periodic or aperiodic basis. Calibration may be performed in any suitable manner. For example, calibration may occur in an automatic manner by measuring respective distances from a common element, or by using signal processing to align a common element in 2D images captured by each of the medical devices 100_{A}, 100_{B}. It is envisioned that each of the medical devices 100_{A}, 100_{B} may communicate with the receiver 10 (FIG. 1) using a communications protocol in a multiplexed or other manner, as understood in the art. It is envisioned that the medical devices 100_{A}, 100_{B} may be time synchronized such that data captured by each of the medical devices 100_{A}, 100_{B} may be synchronized by the receiver 10.

In one embodiment, the medical device 100_{A}, 100_{B} and the receiver 10 may be connected by a bi-directional communications channel, e.g., a Bluetooth communications channel. In those embodiments where the medical device(s) 100_{A}, 100_{B} are configured with an audio speaker, the patient P (FIG. 1) may be able to play music or speak with the fetus F via the receiver 10 (e.g., mobile phone) and the medical device(s) 100_{A}, 100_{B}. It is further envisioned that the medical devices 100_{A}, 100_{B} may be configured and adapted to interface with the receiver 10 to facilitate real time, or near real time, video and/or audio receipt and transmission to enable the patient P to observe and/or engage with the fetus F, e.g., on a smart phone.

In certain embodiments, the disclosed medical device, e.g., the medical device 100, may include a surgical navigation system, such as the superDimension™ navigation system, and may include a separate processing unit to receive and/or transmit data, such as, for example, lung development data. For example, in the context of a surgical lung surgery on a pregnant female, the medical device, e.g., the medical device 100 (FIGS. 1, 2), may be used to relay information pertinent to the fetus F to the surgeon, including indicators of fetal stress, such as a change in fetal heart rate, or information as to how the fetus F is reacting to various anesthetics administered to the patient P.

As previously described, although shown in different embodiments, the various sensing devices in the different medical devices 100, 200, and 300 may be combined into a medical device, e.g., a single capsule, which may be enlarged to support additional sensing components. If multiple sensing devices are utilized in a single medical device, then each of the sensing components may be operated in combination or independently. In other words, first and second medical devices, 100A, 100B, respectively, may each include various sensing devices, as described herein.

In various embodiments, it is envisioned that the first and/or second medical devices may be programmable to collect and transmit various types of data. For example, the data may be audio and/or video data depending on the type(s) of sensors in the medical device available for sensing a fetus. In one embodiment, the video signal may result from an ultrasound transducer configured to generate and sense ultrasound signals.

It is envisioned that the transmission of data from the medical device 100 to the receiver 10 may be either direct and/or indirect. For example, collected data may transmitted from the medical device 100 to another party, individual, corporation, service provider, or device, where the data may be processed, interpreted, analyzed, configured, and the like, and thereafter, transmitted to the receiver 10. As an example, the data may be transmitted via a WiFi device (not shown) through the Internet or other network (not shown) to a remote server (not shown), and back to the receiver 10 via the Internet or other communications network. The data may be processed by the server to generate image data or any other data, such as annotated or highlighted data to show and/or identify certain features. In one embodiment, the data at the server may be accessed by a 3rd party, such as a doctor or other medical provider. Still yet, the server may operate as a cloud storage system for the user of the receiver 10.

### Methods of Use

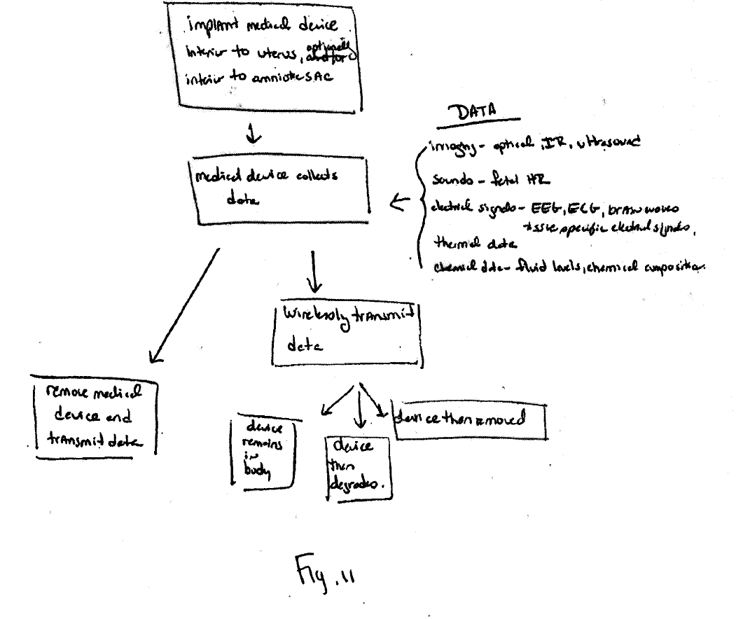

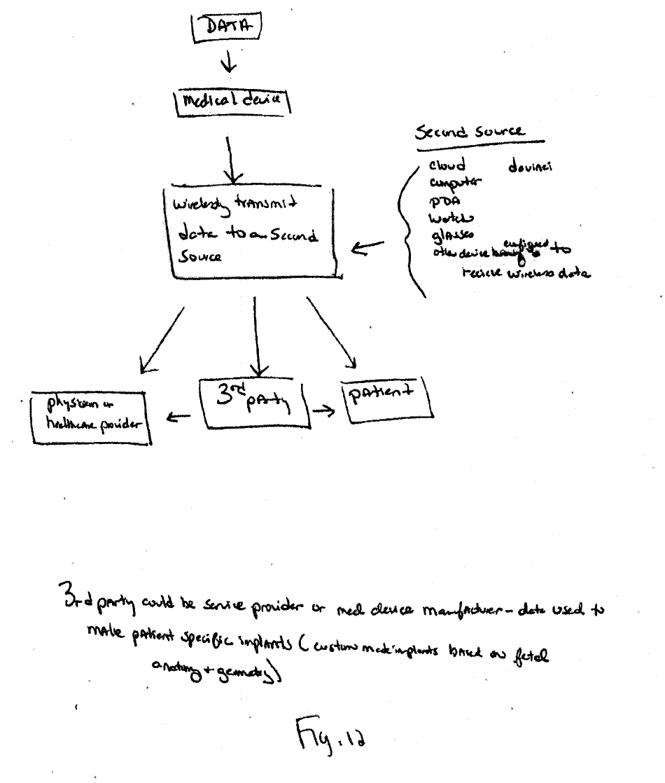

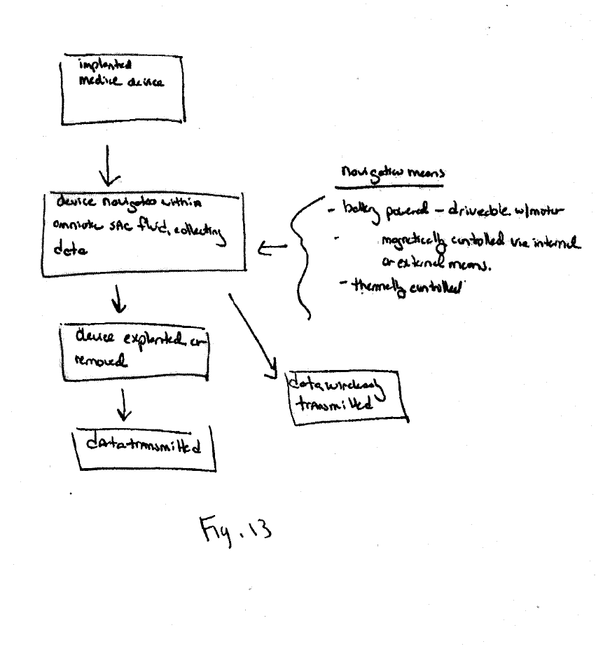

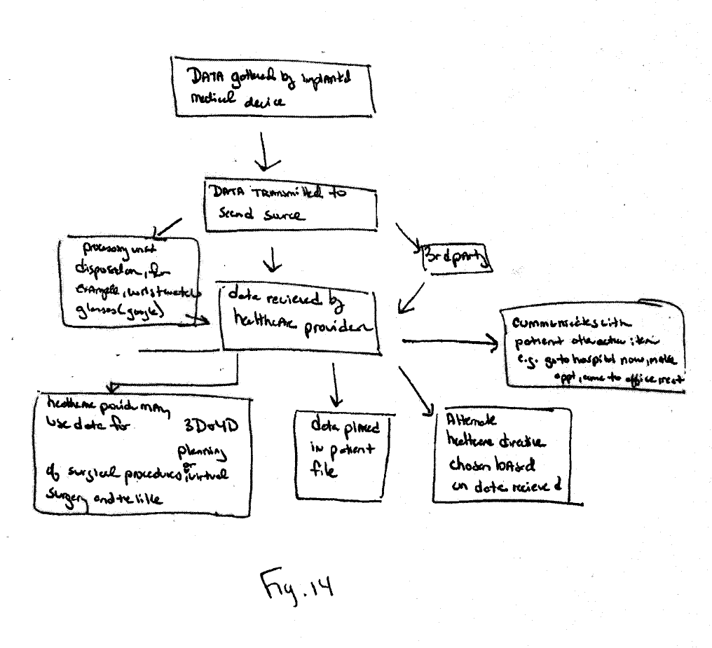

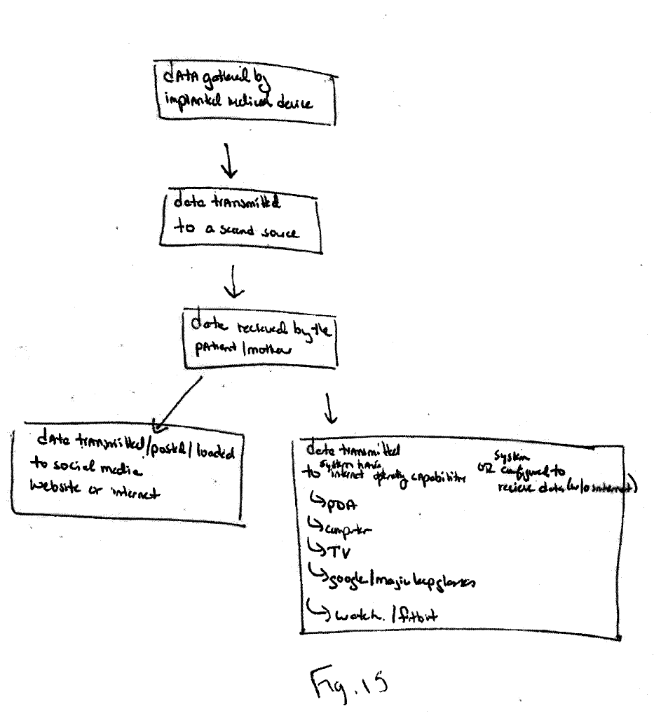

With reference now to FIGS. 1, 2, and 10-15, various methods of using the medical device 100 will be discussed. Initially, the medical device 100 is inserted into the patient P. For example, it is envisioned that the medical device 100 may be configured and dimensioned for insertion through a needle (not shown).

After insertion into the patient P, e.g., into the patient's uterus U or the amniotic sac S, if necessary or desirable, the medical device 100 can be implanted or secured to one or more internal structures. For example, the anchoring structure(s) 102 (FIG. 2) may be affixed to an internal wall of the patient P's uterus U or to an exterior surface of the amniotic sac S, e.g., via suturing. Alternatively, the medical device 100 may be unsecured to any internal structure, e.g., to facilitate remote controlled navigation of the medical device 100, for example, within the patient's uterus U or the amniotic sac S, in the manner discussed above.

Thereafter, the medical device 100 can be used to collect and/or record fetal data and/or data pertinent to the patient P. For example, the medical device 100 may be utilized to collect ultrasound images of the fetus F, measure the heart rate of the fetus F, or take photographs or video of the fetus F, as discussed above. Additionally, or alternatively, the medical device 100 may be used to collect bodily fluid samples, tissue samples, etc., either from the patient P or the fetus F, for analysis, e.g., via storage within the reservoir(s) 426 (FIG. 8).

Once collected and/or recorded, if necessary or desirable, the data can be transmitted, e.g., wirelessly, to the receiver 10 (FIG. 1) for subsequent use or analysis in the manner discussed above. In one embodiments of the disclosure, it is envisioned that the receiver 10 may be located externally of the patient P. Alternatively, the receiver 10 may be located internally, within the patient P, in the form of a second medical device that is inserted during the course of a medical or surgical procedure, and subsequently removed following receipt of the data such that the data can be downloaded. In such embodiments, like the first medical device, e.g., the medical device 100, the second medical device may be configured and dimensioned for implantation into the patient P's uterus, the amniotic sac S, or any other suitable internal structure, organ, or the like, either partially or entirely, on either a permanent basis, or a temporary basis, and thus, may include or be used with anchoring structure(s), e.g., mechanical and/or chemical, as discussed above in connection with the medical device 100.

In an alternate method of use, once the necessary or desired fetal data has been collected, the medical device 100 can be removed from the patient P, and placed in communication with the receiver 10 such that the fetal data can be downloaded. For example, the medical device 100 can be wirelessly connected to the receiver 10, or the medical device 100 and the receiver 10 can by physically connected, e.g., via docking.

### Examples

Medical device 100 may include hardware and software for bi-directional communication with a receiver (processor, transmitter, etc.). The medical device may be configured for collecting ultrasound data and wirelessly transmitting to a receiver, such as a desktop computer. The medical device may be implanted within the patient during an outpatient procedure. Once implanted, navigation of the medical device may be externally controlled to navigate the medical device within the amniotic sac of the patient. As the medical device moves throughout the amniotic sac, the medical device collects various fetal images, recordings (video), and fetal heart rate and wirelessly transmits them to a receiver. The ultrasound data may then be analyzed by a medical specialist (e.g., ultrasound technician, physician) and then placed in the electronic records of the patient.

In another example, the medical device 100 collects ultrasound data (2D, 3D, 4D and CW Doppler), and further includes a sensing system for measuring intrauterine blood flow. Ultrasound and blood flow data may be continuously or intermittently sent via wireless communication to a receiver. The receiver may include baseline data and computing capabilities to recognize/process normal data. When receiver recognizes data is outside the normal/acceptable/pre-programmed levels, a communication (e.g., email, text, phone call, etc.) may be sent to medical specialist and patient that alerts the patient to immediately check in with medical provider and/or check into hospital.

In another example, the medical device 100 is implanted into the amniotic sac and includes biosensors 528 to measure chemical compositions of fluids contained within an amniotic sac. The medical device further includes an anchoring structure such as micro-hooks to attach the medical device to the placenta. The medical device 100 may include a receiver 10 to wirelessly transmit data including fetal kidney function to a second receiver outside or external to the patient.

In yet another example, a 2-way communication including audio and/or video between the patient and fetus may be provided by audio and video-for patient and fetus. In one embodiment, an app may be executed on a mobile device (e.g., tablet, smartphone watch, or otherwise). The 2-way communication may be initiated by either the fetus or user, as further described herein. Additional features, as also further described herein, may be provided by the app to provide for additional entertainment and/or medical examination value to the user and/or medical professional.

4. In another example, a medical device 100 is inserted into an amniotic sac via a small needle. The medical device 100 then is navigable inside the amniotic sac via a motor and collects tissue samples from inside the amniotic sac. Other data including fetal images are collected by the medical device while navigating inside the patient. Once data collection is complete, the medical device 100 is removable from the amniotic sac utilizing a retrieval device, which may be the same needle as insertion. The medical device 100 then transmits the data including tissue samples and fetal images to a healthcare provider.

5. In another example, a medical device 100 is implanted intravaginally on a uterine wall of a patient. The medical device 100 is configured as a patch and may be affixed to the uterine wall via an adhesive. The medical device 100 includes sensing means to detect, monitor, and measure vaginal bleeding in additional to sensing means to monitor the fetal heart rate as described hereinabove. Once the vaginal bleeding has stopped, the medical device 100 may be removed from the uterine wall by a healthcare provider.

6. In another example, a medical device system having a first medical device and a second medical device is implanted at a spaced distance apart within a patient. At least one of the medical devices includes a receiver configured for bi-directional communication. In this particular example, the first medical device may be implanted on a temporary basis, for example for a few months to collect and transmit data (then removed), while the second medical device containing the receiver may be implanted for a longer amount of time to enable continuous or intermittent bi-directional communication.

### Fetus Entertainment System

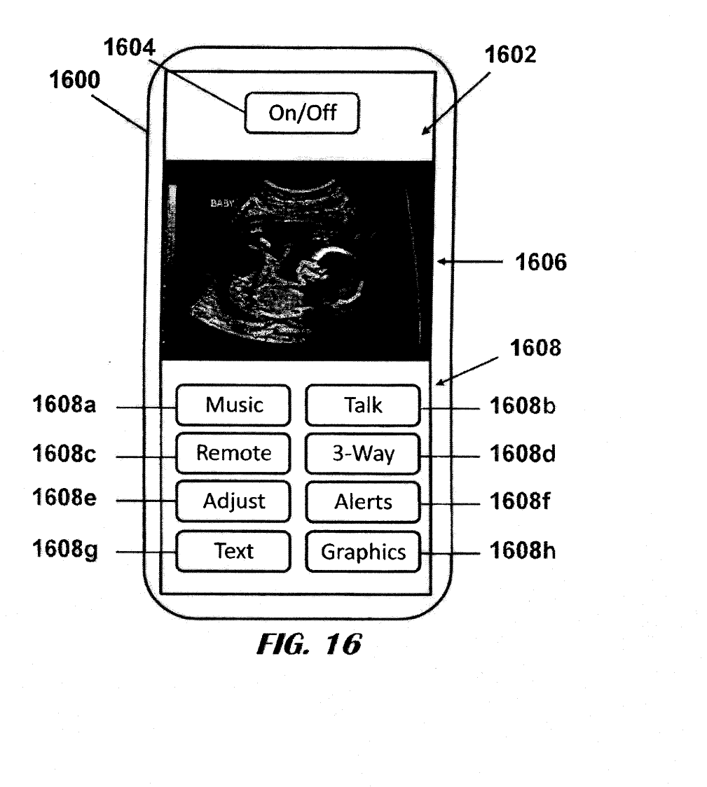

With regard to FIG. 16, a screenshot of an illustrative electronic device 1600 executing an illustrative user interface 1602 for communicating with and controlling a medical device, such as medical device 100, is shown. The electronic device 1600, in this case, is a mobile device, such as a smartphone. Software, such as a mobile app, may be executed by the electronic device 1600. Other electronic devices, such as tablets, laptops, desktops, or other electronic devices, may also be utilized to communicate with the medical device locally via a direct communications link (e.g., Bluetooth®) or remotely via a local or wide area network communications link. Communications between the electronic device and medical device may include content and/or control signals. As shown, the user interface 1602 may include a number of selectable soft-buttons, image(s), or otherwise.

In one embodiment, the user interface 1602 may include an “on/off” soft-button 1604 that enables a user to turn on and off the medical device. When the medical device is turned on, in the case of imaging, such as ultrasound imaging, an image 1606 of the fetus may be displayed on the user interface 1602. Depending upon the sensor type, the image may be varied. For example, if a thermal sensor is used, then thermal images may be displayed. Still yet, if an audio sensor is used, then no image may be displayed, but rather audio signals may be played through a speaker of the electronic device 1600. The image 1606 may be zoomed in and out using multiple fingers, via a GUI element, such as a zoom bar, or otherwise.

The user interface 1602 may include one or more soft-buttons 1608 that enable to user to operate a medical device, manage communications, generate control signals, or otherwise. A “Music” soft-button 1608a may enable a user to communicate music from the mobile device to the medical device (or multiple medical devices if multiple medical devices have been implanted) wirelessly or via a wire depending on the configuration of the medical device with respect to the mobile device 1600.

In response to the user selecting the “Music” soft-button 1608a, a user may be prompted with another user interface (not shown) that enables the user to select music. The music may be music stored by a user on the electronic device 1600, and, in one embodiment, interface with a music program, such as iTunes, as understood in the art, to select music to play to the fetus.

A “Talk” soft-button 1608b may enable the user to talk to the fetus via the medical device(s). For both the music and talk features, the medical device(s) are to be configured with a speaker so that music and/or talk may be communicated to the fetus via the medical devices.

A “Remote” soft-button 1608c may enable a user to allow for a 3rd party, such as a medical professional monitoring the fetus, to remotely access the medical device. In response to the user selecting the “Remote” soft-button 1608c, the user may be prompted approve the 3rd party. In one embodiment, the “Remote” soft-button 1608c may cause another user interface or pop-up window to be displayed so that the user may call a 3rd party's telephone. Once the user provides access to the 3rd party, the user need not participate in the access to the medical device (i.e., the 3rd party may either access the medical device via another communications channel independent of the electronic device 1600 or access the medical device via the electronic device 1600, but without further user interaction so long as the electronic device 1600 remains in communication with the medical device.

A “3-Way” soft-button 1608d may be provided to enable a user to conduct a 3-way video call with another person on a mobile device. The 3-way video chat may enable a user to include a video of the fetus with another person, such as the father of the fetus. Other examples are also possible.

An “Adjust” soft-button 1608e may enable a user to move or reorient a camera or other sensing device in the medical device. In response to selecting the “Adjust” soft-button 1608e, a user interface may be displayed that enables the user to reposition a sensor, such as a camera, by moving his or her finger on the screen of the mobile device, for example, until the sensor is pointed in the direction desired by the user. In one embodiment, the user may zoom in and out of the image 1606, and the sensor may be reoriented or repositioned based on the zoom or movement commanded by the user by interacting with the image 1606. In an alternative embodiment, an application being executed by the electronic device 1600 may operate to automatically reorient the sensor of the medical device. In yet another embodiment, responsive to selecting the “Adjust” soft-button 1608e may cause another user interface to be displayed that includes selectable elements, such as preselected locations to orient or reposition the medical device. For example, preselected locations may include heart, kidneys, liver, or any other organ or location of the fetus that can be automatically identified by signal and/or image processing.

An “Alerts” soft-button 1608f may enable a user to select and/or establish alerts to be created in response to a measurement outside of a particular range. For example, in the case of measuring a heartbeat of a fetus using the medical device, the medical device or software executing on the electronic device 1600 may cause an alert to be generated on the electronic device. For example, the alert may be a notification displayed on the electronic device 1600 and/or audio/vibration notification so that the user is able to respond to a problem being sensed. The alert may also cause the electronic device to send an alert or prompt the user to send an alert to a doctor about a problem identified by the medical device. The alert may include specific information (e.g., “fetus heart rate low,” photographic or video of a specific organ operationally with graphics, such as scale, may be sent with an alert, or any other information may be included) that is automatically generated by the software being executed by the electronic device 1600. A wide variety of alerts may be available.

In addition to per se alerts, the “alerts” soft-button may enable a user to set up a fetus communication initiation, whereby a fetus may activate a “call” or other communication to the electronic device 1600 of the mother by performing an action, such as waking up, turning to face the medical device, smiling, yawning, having a heartbeat increase above a threshold level, or otherwise. By creating such a fetus communication initiation, the mother may capture moments that are otherwise difficult to capture and increase the experience of the mother.

A “Text” soft-button 1608g may enable the user to include text on the image 1606 for fun or for information purposes. For example, a user may add text of the fetus' name, current date, expected delivery date, current weight, or any other information to the image 1606. In one embodiment, pre-established messages, such as “Happy Birthday to me!”, may be available for a user to select and place on the image 1606. Other messages, such as “I'm asleep,” “I'm awake,” “Good morning mommy,” “I'm happy,” “I'm ready to meet you,” “See you soon,” and “There's two of us!” may be available, as well.

A “Graphics” soft-button 1608h may provide for a variety of pre-established graphics or enable the user to free-hand graphics on the image 1606. For example, the graphics may include emoticons, images of items (e.g., hats, smiles, faces, clothing, birthday cakes, etc.) that the user may enjoy adding to the image 1606. Other medically relevant images may be available, such as length scale, distance measurement elements to measure head size, kidney size, or other organ sizes.

Although not shown, a soft-button may provide for the user to send a copy of the image with the text (or other graphics) via a message, such as a text message, email, or save to the electronic device 1600.

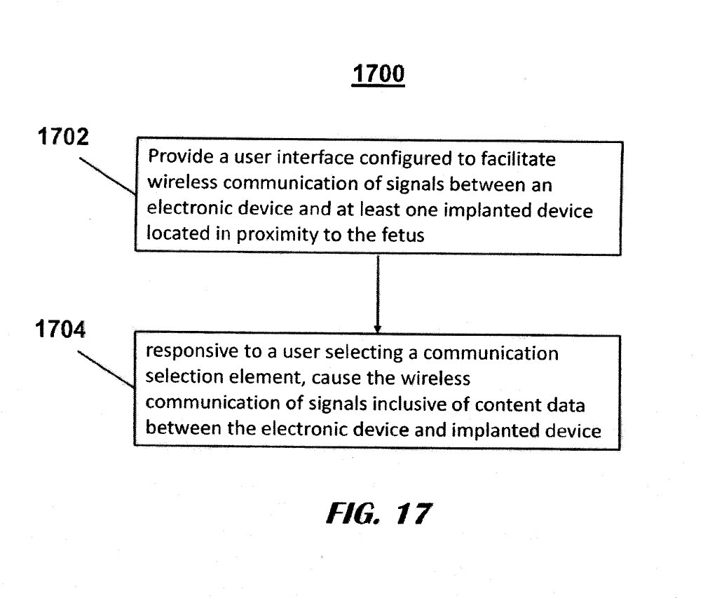

With regard to FIG. 17, a flow diagram of an illustrative process 1700 for communicating with a fetus is shown. The process 1700 may include providing, on an electronic device, a user interface configured to facilitate wireless communication of signals between the electronic device and at least one implanted device located in proximity to the fetus. At step 1704, responsive to a user selecting a communication selection element, signals inclusive of content data may be caused to be wirelessly communicated between the electronic device and implanted device.

In one embodiment, the wireless communication of signals may include image data of the fetus captured by the implanted device to the electronic device. The process may include communicating video data captured by the implanted device to the electronic device. The process may include communicating audio data from the fetus captured by the implanted device to the electronic device. The process may include communicating audio data from the electronic device to the implanted device for audible output by the implanted device. The process may further include providing a selection element on the user interface that enables the user to select music from a selectable list of music and responsive to the selection of music, causing the music to be communicated from the electronic device to the implanted device. The process may further include providing a selection element on the user interface that enables the user to select a video chat user interface, and causing a 2-way communication that enables the electronic device to receive video data from the implanted electronic device and to communicate audio data from the user via the electronic device to the implanted electronic device. The process may include communicating video data captured by first and second implanted devices to the electronic device, processing the video data received from the first and second implanted electronic devices to produce 3D video data, and displaying the 3D video data on the electronic device. In an embodiment, 2-way communication may be between two or more devices, such that a conference communication between three or more devices, including the implanted device(s), may be performed. In the event that a stereoscopic pair of implanted medical devices are implanted, a video chat (or image sharing) may provide for 3D image(s) to be presented to a user, where the 3D image(s) may be formed by an implanted device or electronic device(s), such as a mobile telephone.

In one embodiment, the process may further include providing a graphical user element that enables a user to enter information and select pre-established graphics, and displaying the entered and/or selected information or selected pre-established graphics on an electronic display of the electronic device in relation to an image of the fetus from content data received from the implanted device. The process may further include displaying the information or graphics includes displaying text and/or graphical images. In one embodiment, the process may further include establishing preset messages, and responsive to determining that a signal received from the implanted device corresponds to a preset message, cause the preset message to be displayed on an electronic display of the electronic device.

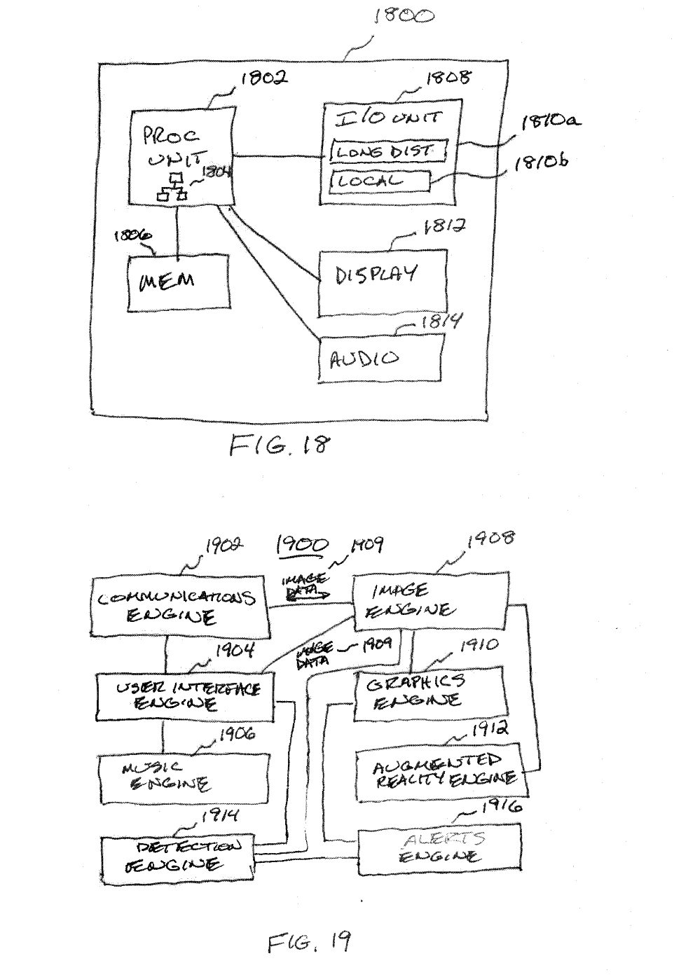

With regard to FIG. 18, a block diagram of an illustrative electronic device 1800 configured to support functionality for communicating with a fetus as described herein is shown. The electronic device 1800 includes a processing unit 1802 that executes software 1804. The software 1804 may provide for a variety of functions, as described with regard to FIGS. 16 and 17, for example, to enable a user to interface with the electronic device 1800 when sensing, monitoring, measuring, communicating with, and/or entertaining a fetus. The processing unit 1802 may be in communication with a memory unit 1806 configured to store software and data (e.g., image content), and input/output (I/O) unit 1808 that includes a long distance transceiver 1810a and local transceiver 1810b. The long-distance transceiver 1810a may be configured to communicate over a telecommunications or other communications network. The local transceiver 1810 be may be configured to communicate over a local communications channel, such as Wi-Fi, Bluetooth, or otherwise. The processing unit 1802 my further be in communication with an electronic display 1812, which may be a touch screen, that enables the user to interface with a user interface displayed on the electronic display 1812. An audio device 1814, such as a speaker, may be in communication with the processing unit 1802 to enable audio signals, such as motion sounds, music, or other audible content to be played for a user.

With regard to FIG. 19, a set of engines 1900 that may be executed by the processing unit 1802 is shown. The engines may include a communications engine 1902 that may be utilized to bi-directionally communicate via the I/O unit 1808 for long distance and/or local communications. The local communications may include communicating with a medical device that may be implanted, as previously described herein. The communications engine 1902 may be configured to communicate control commands and/or data, such as image, audio, or other data, as previously described herein. The commands may include positioning commands, ON/OFF commands, or any other commands that may be used to control functionality of a medical device.

A user interface engine 1904 may be configured to manage a user interface, such as the user interface 1602 of FIG. 16. The user interface engine 1904 may be configured to receive a response to selections or other user interface requests by a user of the user interface 1602. It should be understood that the user interface 1602 is illustrative, and that many alternative and/or additional features of the user interface 1602 may be provided and managed by user interface engine 1904.

A music engine 1906 may be configured to engage with music stored on an electronic device being utilized by a user. For example, if the user has downloaded music to the electronic device, the music engine 1906 may engage with the music directly or via a music manager that is also being executed by the electronic device. Moreover, the music engine 1906 may be configured to access music files available in a music library on a communications network, as understood in the art. The user interface engine 1904 and/or music engine 1906 may be configured to present a selectable list of music for wireless communication to an implantable device.

An image engine 1908 may be configured to enable the user to capture and display photographs and/or video. In an embodiment, the image engine 1908 may be in communication with the communications engine 1902, and be configured to receive image data 1909 being captured by a medical device implanted near a fetus. The image engine 1908 may thereafter be configured to process and communicate the image data to the user interface engine 1904 for display on a user interface for a user. In an embodiment, a graphics engine 1910 may be configured to enable a user to display various graphics on the image data. For example, the graphics engine 1910 may be configured to display text and/or graphics (e.g., hats, rattlers, cartoon characters, balloons, faces, etc.). The graphics engine 1910 may be configured with pre-established graphics (e.g., characters, items, such as ties, bows, dresses, etc., emoticons, emojis, text (e.g., happy birthday) or graphic (e.g., “dream cloud”) messages, or other static or dynamic items) that are selectable by the user or automatically selectable by the user interface engine 1904 or other engine.

The image engine 1908 may further be in communication with an augmented reality engine 1912 that enables the user to place characters (e.g., Pokémon) or other augmented reality images, audio, or other augmented reality features, as understood in the art. For example, the augmented reality engine 1912 may be configured to play a swishing sound, kicking sound, yawn sound, snoring sound, for example, if the medical device and/or engines 1900 determine that a fetus moves, kicks, yawns, or sleeps, respectively. It should be understood that alternative communication is configurations may be utilized, such as the graphics engine 1910 and augmented reality engine 1912 may be a communication directly with the user interface engine 1904.

In an embodiment, the engines 1900 may include a detection engine 1914 configured to receive and process image, acoustic, ultrasound, audible, and/or other sensed signals from an implanted medical device. The processing may include image processing, signal processing, or other processing to determine state (e.g., position, asleep, awake, kicking, smiling, etc.), and use that information to generate an alert by an alerts engine 1916, generate an image (e.g., graphic or text), generate a sound (e.g., yawn), or otherwise. The detection engine 1914 may communicate with the user interface engine 1904 or other engine (e.g., image engine 1908) to receive sensory data and communicate information to be displayed or otherwise communicated to a user via the user interface.

Although these inventions have been disclosed in the context of certain preferred embodiments and examples, it will be understood by those skilled in the art that the present inventions extend beyond the specifically disclosed embodiments to other alternative embodiments and/or uses of the inventions and obvious modifications and equivalents thereof. In addition, while several variations of the inventions have been shown and described in detail, other modifications, which are within the scope of these inventions, will be readily apparent to those of skill in the art based upon this disclosure. It is also contemplated that various combination or sub-combinations of the specific features and aspects of the embodiments may be made and still fall within the scope of the inventions. It should be understood that various features and aspects of the disclosed embodiments can be combined with or substituted for one another in order to form varying modes of the disclosed inventions. Thus, it is intended that the scope of at least some of the present inventions herein disclosed should not be limited by the particular disclosed embodiments described above.

What is claimed is:

1. A method of monitoring a fetus in utero, comprising:
implanting a medical device at a uterus of a patient;
collecting fetal data using the medical device; and
transmitting the fetal data from the medical device to a receiver.

2. The method of claim 1, further including securing the medical device within the patient's uterus.

3. The method of claim 2, wherein securing the medical device includes securing the medical device to an interior wall of the patient's uterus.

4. The method of claim 1, further including positioning the medical device within an amniotic sac.

5. The method of claim 1, further comprising navigating the medical device within the amniotic sac.

6. The method of claim 1, further including securing the medical device to an exterior surface of an amniotic sac.

7. The method of claim 1, wherein transmitting the fetal data includes wirelessly transmitting the fetal data to the receiver.

8. The method of claim 7, wherein wirelessly transmitting the fetal data to the receiver includes wirelessly transmitting the fetal data to a location external of the patient's uterus.

9. The method of claim 8, wherein wirelessly transmitting the fetal data includes wirelessly transmitting the fetal data to a receiver selected from the group consisting of a computer, a cloud, a robotic operating system, and a portable electronic device.

10. The method of claim 1, further including inserting the receiver into the patient, wherein transmitting the fetal data includes transmitting the fetal data to the inserted receiver.

11. The method of claim 10, further including removing the receiver from the patient.

12. The method of claim 11, further including downloading the fetal data from the receiver.

13. The method of claim 1, wherein collecting the fetal data includes capturing at least one image of the fetus, and transmitting the fetal data includes transmitting the at least one image to the receiver.

14. The method of claim 14, wherein capturing the at least one image of the fetus includes capturing one or more still photographs of the fetus.

15. The method of claim 14, wherein capturing the at least one image of the fetus includes recording video of the fetus.

16. The method of claim 1, wherein collecting the fetal data includes collecting data pertinent to health of the fetus.

17. The method of claim 17, wherein collecting the fetal data includes collecting data selected from the group consisting of fluid levels, fluid compositions, fetal sounds, intra-uterine images, electrical signals, and combinations thereof.

18. The method of claim 17, wherein collecting the fetal data includes measuring anatomical structures.

19. The method of claim 17, wherein collecting the fetal data includes collecting a biological sample from the fetus, amniotic fluid, amniotic sac, placenta, or uterine wall.

20. The method of claim 19, wherein collecting the biological sample includes collecting the biological sample selected from the group consisting of tissue, blood, and amniotic fluid.

21. A medical device for monitoring a fetus in utero, comprising:
a securing member configured to secure the medical device to a uterus of a patient;
at least one sensing element configured to collect fetal data;
a transmitter configured to transmit the collected fetal data from the medical device to a receiver; and
a receiver configured to receive communications signals from an electronic device.

22. The medical device of claim 21, wherein the medical device further includes a second receiver configured to receive communications signals from a second electronic device.
